(19) United States
(12) Patent Application Publication
(10) Pub. No.: US 2025/0120817 A1
(43) Pub. Date: Apr. 17, 2025

(54) ARTIFICIAL HIP JOINT

(71) Applicants: Boo Rak LEE, Gimpo-si (KR); Dong Ki LEE, Gimpo-si (KR)

(72) Inventors: Boo Rak LEE, Gimpo-si (KR); Dong Ki LEE, Gimpo-si (KR)

(21) Appl. No.: 18/718,090

(22) PCT Filed: Dec. 12, 2022
(86) PCT No.: PCT/KR2022/020159

§ 371 Date: Jun. 10, 2024

(30) Foreign Application Priority Data
Dec. 10, 2021 (KR) 10-2021-0176497
Dec. 10, 2021 (KR) 10-2021-0176500
Dec. 10, 2021 (KR) 10-2021-0176501

(51) Int. Cl. A61F 2/34; A61F 2/36; A61L 27/10; A61L 27/42

(52) U.S. Cl. CPC A61F 2/34; A61F 2/3609; A61L 27/10; A61L 27/42

(57) ABSTRACT

Disclosed is an artificial hip joint device. This artificial hip joint comprises a head and a liner, wherein at least one among the head and the liner is made of a composite containing zirconia as a first phase and alumina as a second phase, and the content of the first phase and the content of the second phase may be in the range of 50-75 wt % and 25-50 wt %, respectively.

### TECHNICAL FIELD

The present invention relates to an artificial hip joint, and more particularly, to an artificial hip joint made of a biocompatible ceramic material that is usable for a damaged human or animal femoral and having improved wear resistance.

### BACKGROUND ART

End parts of bones that make up joints are made of soft, gliding hyaline cartilages. The hyaline cartilage is like a cushion that can withstand external forces or severe pressure and allows bones to easily move without pain. Such a joint is covered with a soft joint capsule having blood veins. There is a synovial membrane in the joint capsule, which creates a slippery joint fluid to prevent friction or wear and tear of the joint.

The femoral joint, which is a kind of joint, is protected by a thick joint membrane, but in a case where a considerable external force is applied thereto, a failure may occur around the joint, and in a case where a hip joint is weakened by osteoporosis, damage such as a hip joint failure may occur. The hip joint failure includes a femoral neck failure and an acetabular failure. Further, degenerative arthritis or blood circulation disorder may cause avascular necrosis.

In severe cases of damage due to the hip joint failures or avascular necrosis, it is not possible to expect complete recovery with bone traction, and it is necessary to perform artificial joint replacement surgery for replacing an original hip joint with an artificial joint.

An artificial hip joint can replace a patient's damaged hip joint to allow normal activities, but the artificial joint has a considerable difference in performance depending on what material it is made of.

In general, artificial joints are made of metal materials such as stainless steel, cobalt chromium alloy, and titanium alloy or special plastic materials such as polyimide.

The metal materials such as stainless steel or cobalt chrome have a disadvantage in that uneven wear occurs during use. The plastic materials have a disadvantage in that fragments generated by abrasion are eaten by macrophages to cause an immunological action of destroying the macrophages, which results in various tissue-dissolving substances, especially, substances which activate osteoclasts, thereby causing bone dissolution.

Accordingly, in such artificial joints made of metal and plastic materials, dislocation occurs over time due to wear of the artificial hip joint, strong external physical forces, and structural defects of the artificial hip joint, which causes the necessity of revision surgery.

Further, a head and a liner of the artificial hip joint are used as elements for rotation of the joint, in which the head has a spherical shape and the liner has a shape corresponding to the head. The exterior of the head is movably coupled to the interior of the liner. That is, while the artificial hip joint is performing a joint function, the head and the liner continuously come into frictional contact, and thus, they may be easily worn when used for a long time.

### DISCLOSURE

### Technical Problem

The present invention has been made in view of the above problems, and it is an object of the present invention to provide an artificial hip joint having a structure capable of minimizing abrasion of a head and a liner even when used for a long time.

Another object of the present invention is to provide an artificial hip joint capable of easily achieving fusion between patient cells and the artificial hip joint and having excellent implant reliability, structural stability, biocompatibility, and the like.

### Technical Solution

In accordance with an aspect of the present invention, the above and other objects can be accomplished by the provision of an artificial hip joint including a head and a linear, in which at least one of the head or the liner is formed of a composite containing zirconia as a first phase and alumina as a second phase, the content of the first phase is in a range of 50 to 75% by weight, and the content of the second phase is in a range of 25 to 50% by weight.

The content of the first phase may be in a range of 65 to 70% by weight, and the content of the second phase may be in a range of 30 to 35% by weight.

In accordance with another aspect of the present invention, there is provided an artificial hip joint including a head and a linear, in which at least one of the head or the liner may be formed of a composite containing silicon nitride as a first phase and zirconia as a second phase.

The content of the first phase may be in a range of 10 to 90% by weight, and the content of the second phase may be in a range of 90 to 10% by weight.

The particle size distribution of the composite may be 60% or more, and the average particle size of the composite may be in a range of 150 nm to 200 nm.

In accordance with a further aspect of the present invention, there is provided an artificial hip joint including a head and a liner, in which the head may contain 100% by weight of silicon nitride.

The particle size distribution of the silicon nitride may be 60% or more, and the average particle size of the silicon nitride may be in a range of 150 nm to 200 nm.

Any one of the head and liner may be configured so that SARS-CoV-2 virus is inactivated.

At least one of an inner surface of the liner or an outer surface of the head facing the liner may be formed with a plurality of dimples having a predetermined size and a predetermined depth.

The diameter or lateral size of the maximum micropore among the dimples may be in a range of 100 μm to 400 μm, the ratio of the depth to the lateral size may be in a range of 0.04 to 0.2, and the area density of the micropores may be 1% to 30%.

The diameter or lateral size of the minimum micropore of the dimples may be in a range of 50 μm to 100 μm, the ratio of the depth to the lateral size may be in a range of 0.05 to 0.18, and the area density of the micropores may be 1% to 30%.

Each of the head and the liner may satisfy all of Conditional Expression 1.

RH>La1 <Conditional expression 1>

RH=La2

Ra≤0.05 μm

Here, RH is the outer diameter of the head, La1 is the groove diameter of the top inner surface of the liner 20, La2 is the groove diameter of the inner surface of the liner 20, and Ra is an arithmetic mean surface roughness.

The head and the liner have a specific wear rate of 0.5×10-8 mm3/Nm or less, a bending strength of at least 800 MPa or more, a density of 5 g/cm2 or more, and an elastic modulus in a range of 800 to 1000 MPa, and show no fatigue failure cracks.

The head has a surface roughness value of 0.01 μm±50% and a roundness value of 0.1 μm±50%.

Any one of the head and liner may be configured so that as a result a rat macrophage experiment in which the artificial hip joint is contacted with a rat macrophage RAW 264.7 cell line for 24 hours, a cell survival rate of 70% or more is obtained, and in relation to inflammatory response, the amount of production of TNF-α is 300 picograms/ml or less, and the amount of production of IL-1 is 50 picograms/ml or less.

The liner may be formed with a groove portion at an upper apex thereof.

### Advantageous Effects

The artificial hip joint according to the present invention is made of a ceramic material that is a biomaterial, and thus, has excellent strength and wear resistance, as well as high hardness, high temperature resistance, and excellent corrosion resistance.

The artificial hip joint according to the present invention has anti-pathogenic properties and can provide an image without distortion in X-ray, CT and MRI. Further, the artificial hip joint according to the present invention has excellent implant reliability, structural stability, biocompatibility, and the like, and is resistant to viruses such as Corona 19.

In addition, in the artificial hip joint according to the present invention, as the plurality of dimples are formed on the outer surface of the head, an extruded liquid film of a certain thickness is formed on a friction contact surface with respect to the liner, to thereby make it possible to relieve direct rough surface contact to reduce a friction coefficient and a frictional force, thereby enhancing a bearing capacity.

### DESCRIPTION OF DRAWINGS

FIG. 1 is an exploded perspective view of an artificial hip joint according to an embodiment of the present invention.

FIG. 2 is a side sectional view showing a coupled state of the artificial hip joint according to the embodiment of the present invention.

FIG. 3 is a partial sectional view showing a coupling relationship between a liner and a head of the artificial hip joint according to the embodiment of the present invention.

FIG. 4 is a side sectional view of the head of the artificial hip joint according to the embodiment of the present invention.

FIG. 5 is a side view of the head of the artificial hip joint according to the embodiment of the present invention.

FIG. 6 is a side sectional view of the liner of the artificial hip joint according to the embodiment of the present invention.

FIG. 7 is a side view of the liner of the artificial hip joint according to the embodiment of the present invention.

FIG. 8 is a side view showing a coupling relationship between a liner of an artificial hip joint and a head having dimples on its outer surface, according to another embodiment of the present invention.

FIG. 9 is a side view of the head formed with dimples according to another embodiment of the present invention.

FIG. 10 is a partial sectional view of a liner having dimples on its inner surface in an artificial hip joint according to another embodiment of the present invention.

FIG. 11 is a side sectional view showing a coupling relationship in a case where there is a gap between an acetabular cup and a liner of an artificial hip joint, according to an embodiment of the present invention.

FIG. 12 is a side sectional view showing a state in which a head is coupled to a liner having a groove at its upper apex, in an artificial hip joint according to a modified example of the present invention.

FIG. 13 is a side sectional view showing an artificial hip joint in which a liner and a head are coupled, according to another modified example of the present invention.

### BEST MODE

Hereinafter, a configuration of an artificial hip joint including a silicon nitride head according to an embodiment of the present invention will be described in detail with reference to the accompanying drawings.

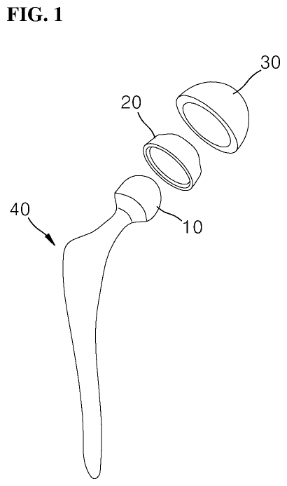

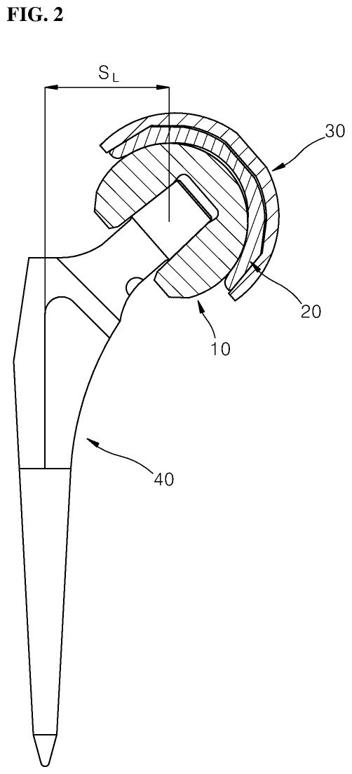

FIG. 1 is an exploded perspective view of an artificial hip joint according to an embodiment of the present invention, and FIG. 2 is a side sectional view showing a coupled state of the artificial hip joint according to the embodiment of the present invention.

Referring to FIGS. 1 and 2, the artificial hip joint includes a head 10 called a femoral head, a liner 20 that is engaged with the head 10, an acetabular cup 30 that surrounds the liner 20, and a stem 40 that is connected to the head 10.

The head 10 and the liner 20 are elements for rotation of the joint, in which the outside of the head 10 is slidably coupled with the inside of the liner 20, and relatively rotates around a rotational axis. The acetabular cup 30 is implanted in a hip bone, and the liner 20 is mounted in the acetabular cup 30. The stem 40 is inserted into a medullary canal of the femur.

Referring to FIG. 2, the artificial hip joint is applied in a state where the head 10, the liner 20, the acetabular cup 30, and the stem 40 are all coupled. A distance (SL) from a handle of the stem 40 to a coupling center of the head 10 may be 30 mm to 36 mm. Further, a rotation angle of the acetabular cup 30 and the liner 20 in a coupled state from the center of the stem 40 may have a range of 62° to 63° left and right. With the effective rotation angle range as described above, a patient can freely move the hip joint without a sense of heterogeneity after implant.

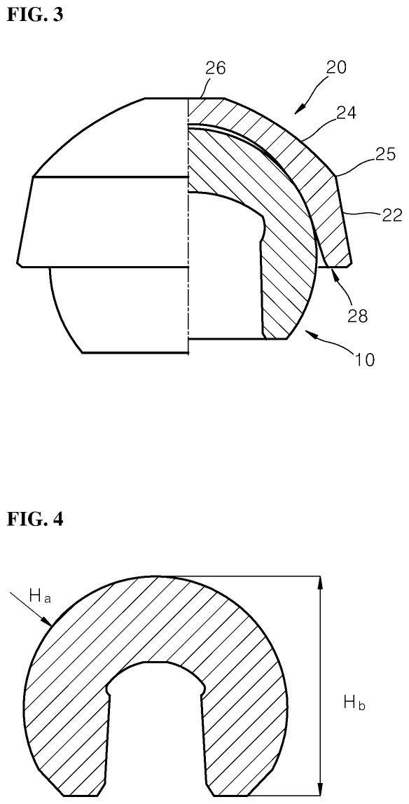

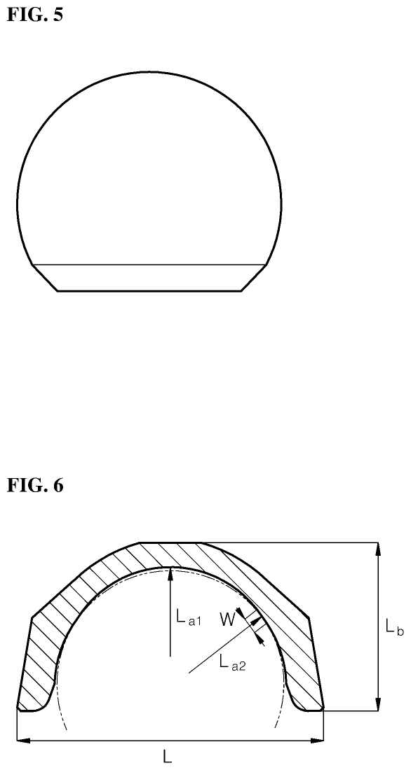

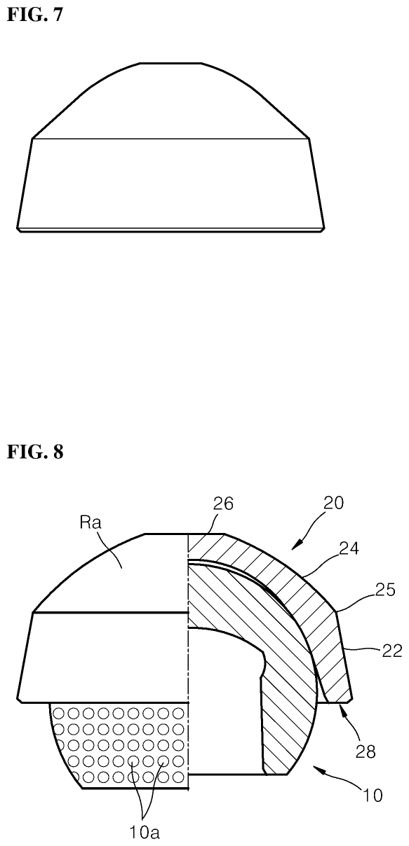

FIG. 3 is a partial sectional view showing a coupling relationship between a liner and a head of the artificial hip joint according to the embodiment of the present invention, FIG. 4 is a side sectional view of the head of the artificial hip joint according to the embodiment of the present invention, FIG. 5 is a side view of the head of the artificial hip joint according to the embodiment of the present invention, FIG. 6 is a side sectional view of the liner of the artificial hip joint according to the embodiment of the present invention, and FIG. 7 is a side view of the liner of the artificial hip joint according to the embodiment of the present invention.

Referring to FIGS. 3 to 7, the liner 20 may include a locking portion 22, a dome portion 24, a change portion 25, and a top portion 26 at an outer surface thereof. The liner 20 may include a concave inner surface configured to be engaged with the head, the outer surface configured to be engaged with the acetabular cup 30, and an edge portion 28 that extends between the inner and outer surfaces. Here, the outer surface may include the locking portion 22 that extends from the edge portion 28, the curved dome portion 24 that extends from the locking portion 22 through the change portion 25, and the top portion 26 that extends from the dome portion 24. Further, the dome portion 24 from an edge point of the top portion 26 to the edge point of the locking portion 22 forms a curved radius.

In terms of dimensions the head 10, a diameter (Ha) of the outer surface may be 28 mm to 40 mm, and a height (Hb) thereof may be 24 mm to 32 mm.

In addition, in terms of dimensions of the liner 20, a groove diameter (La2) of the inner surface may be 28 mm to 40 mm, a height (Lb) thereof may be 19 mm to 25 mm, and a width (L) thereof may be 39 mm to 49 mm.

Each of the head 10 and the liner 20 may satisfy Expression 1 below.

$$RH > La 1 \qquad < Expression 1 >$$

$$RH = La 2$$

$$Ra \leq 0.05 μm$$

Here, RH is the outer diameter of the head, La1 is the groove diameter of the top inner surface of the liner 20, La2 is the groove diameter of the inner surface of the liner 20, and Ra is an arithmetic mean surface roughness.

In a case where the inner diameter of the liner 20 is smaller than the outer diameter of the head 10, the head 10 can easily rotate while contacting the liner 20 without being separated from the liner 20. In particular, the diameter of RH may be approximately 28 mm, 32 mm, 38 mm, or 40 mm, and the diameter of the liner 20 may be approximately 32 mm, 38 mm, or 40 mm. Further, the width (W) of a part in which the outer diameter (RH) of the head 10 and the groove diameter (La2) of the inner surface of the liner 20 are equal to each other, and which is seen as a surface contact part in a cross-sectional view and is seen as a donut-shaped band in a three-dimensional view, may be approximately 2 mm to 3 mm. In a case where the surface contact width (W) is in the range of about 2 mm to 3 mm, the head 10 can rotate naturally without being separated from the liner 20. On the other hand, the surface roughness and roundness of the head 10 and the liner 20 may be adjusted with high precision using precision processing. In this way, since the surface contact between the head 10 and the liner 20 is secured, the specific wear rate and durability may be improved.

That is, the outer diameter (RH) of the head may be larger than the groove diameter (La1) of the upper top inner surface of the liner 20 and equal to the groove diameter (La2) of the inner surface of the liner 20. Accordingly, the head 10 and the liner 20 can be in surface-contact with each other, and thus, it is possible to enhance wear resistance and durability.

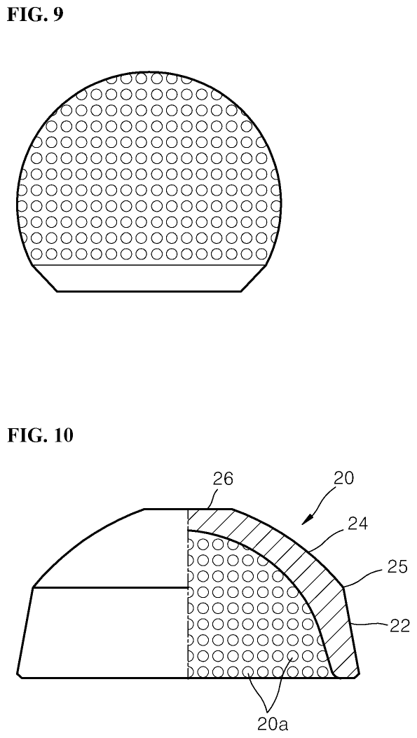

FIG. 8 is a side view showing a coupling relationship between a liner of an artificial hip joint and a head having dimples on its outer surface, according to another embodiment of the present invention, FIG. 9 is a side view of the head formed with dimples according to another embodiment of the present invention, and FIG. 10 is a partial sectional view of a liner having dimples on its inner surface in an artificial hip joint according to another embodiment of the present invention.

Referring to FIGS. 8 to 10, concave or convex dimples 10a or 20a may be formed on at least one of the outer surface of the head 10 or the inner surface of the liner 20. Here, the diameter or lateral size of the maximum micropore of the dimples 10a or 20a may be in a range of 100 μm to 400 μm, the ratio of the depth to the lateral size may be in a range of 0.04 to 0.2, and the area density of the micropores may be 1% to 30%. The diameter or lateral size of the minimum micropore may be less than 50 μm to 100 μm, the ratio of the depth to the lateral size may be in a range of 0.05 to 0.18, and the area density of the micropores may be 1% to 30%.

In a case where the ratio of the depth to the lateral size is less than 0.04 or greater than 0.2, it is difficult to obtain micro roughness when coupling the outer surface of the head 10 and the inner surface of the liner 20, so that the mechanical coupling between the head 10 and the liner 20 may become unstable to cause separation therebetween in long-term bio-implant. In other words, in a case where the ratio of the depth to the lateral size is in the range of 0.04 to 0.2, in the mechanical coupling between the head 10 and the liner 20, it is possible to obtain micro roughness, thereby enhancing compatibility in long-term bio-implant.

As the outer surface of the head 10 or the inner surface of the liner 20 is formed to have the dimples 10a or 20a, the surfaces with the micro dimples can be brought into frictional contact with each other, thereby generating hydrodynamic pressure lubrication.

Further, since an extruded liquid film having a certain thickness is formed between the contact surfaces that form the frictional pair to reduce contact stress on the contact surfaces, it is possible to reduce a friction coefficient and a frictional force, thereby enhancing the bearing capacity. Accordingly, by forming the dimples on the entire surface of the head 10, it is possible to prevent the occurrence of operation troubles due to air compression with respect to the liner 20 that is in contact with the head 10, and to enhance the wear resistance.

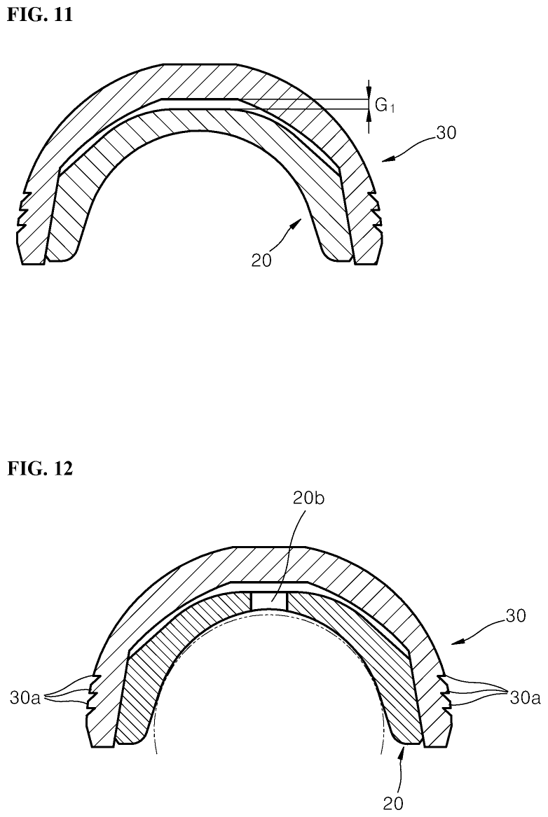

FIG. 11 is a side sectional view showing a coupling relationship in a case where there is a gap G1 between an acetabular cup 30 and a liner 20 of an artificial hip joint, according to an embodiment of the present invention.

Referring to FIG. 11, the gap G1 that decreases in size from the center to the outside is formed between the acetabular cup 30 and the liner 20. The gap G1 between the acetabular cup 30 and the liner 20 after coupling may be approximately 0.48 mm to 1 mm. In a case where the gap G1 is in the range of 0.48 mm to 1 mm, it is possible to provide a plurality of suitable rotational orientations about the rotation axis in a state where the inner surface of the acetabular cup 30 and the outer surface of the liner 20 may be engaged with each other. In a case where the gap G1 is out of the above range, there is a high probability of friction during rotation, and accordingly, wear in long-term implant. In a case where the liner 20 is operated while being inserted in the acetabular cup, in a case where a load is applied to the inserted liner 20, it may cause a load-dependent collapse to deteriorate the coupling force. In a case where there is such a gap between the acetabular cup 30 and the liner 20, it is possible to provide an appropriate coupling force regardless of the load, so that the liner 20 may be coupled with the acetabular cup 30 while securing a strong coupling force with a simple structure. Further, this gap may reduce shocks to the acetabulum when the load is applied to the hip joint.

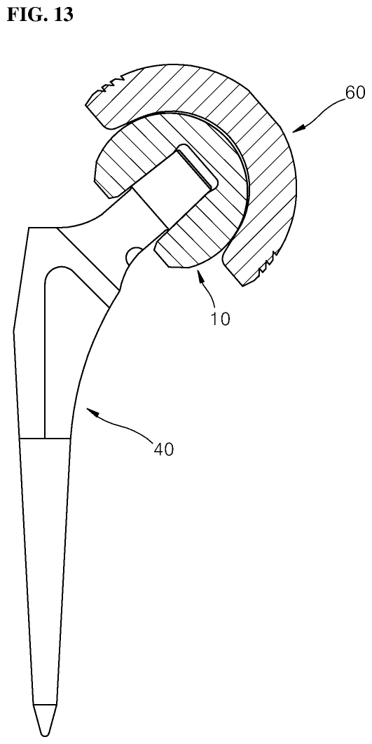

FIG. 12 is a side sectional view showing a state in which a head is coupled to a liner having a groove at its upper apex, in an artificial hip joint according to a modified example of the present invention.

Referring to FIG. 12, in the artificial hip joint including the head 10 and the liner 20, the liner 20 may have a groove portion 20b formed at an upper apex thereof.

The groove portion 20b of the liner 20 can alleviate tension when contacting and coupling with the head 10. Further, a tissue coupling portion 30a may be formed around the outer circumference of the acetabular cup 30. The tissue coupling portion 30a may be formed with a plurality of teeth to increase the coupling force by contacting surrounding human tissues where the artificial hip joint is implanted. Here, each groove portion of the teeth is formed as a groove inclined at 45° or less with respect to the horizontal axis, so that even in a case where the liner 20 rotates, the liner 20 can be coupled to the surrounding tissues and prevented from escaping to the outside. Instead of the shape shown in FIG. 10, various shapes of concavities and convexities may be used.

FIG. 13 is a side sectional view showing a state where all components of the artificial hip joint except for the acetabular cup 30 are coupled to each other, according to a modified example of the present invention.

Referring to FIG. 13, in the artificial hip joint including the head 10 and the liner 20, the liner 20 may be directly implanted into the bone without the acetabular cup 30.

In a case where the surface of the liner 20 is formed with dimples of micropores on the outer surface as in the acetabular cup 30, the liner 20 can be directly implanted into the hip bone without the acetabular cup and the bone grows on the surface of the liner 20, thereby making it possible to simplify the structure.

### Comparative Examples 1 and 2 and Examples 1 to 6 According to Zirconia and Alumina Composites

In Comparative Example 1, a mixed powder was obtained by mixing powders containing 40% by weight of zirconia, 60% by weight of alumina, and less than 1% by weight of other additives (including at least one of titania, calcia, or cerium oxide), with respect to the whole composite material. The mixed powder was molded by uniaxial pressing and isostatic pressing to obtain a green compact of a predetermined shape, and then, the green compact was sintered at 1400° C. for 5 hours under normal pressure in the air. Accordingly, Comparative Example 1 having an average particle size of 100 nm was obtained.

In Comparative Example 2, a mixed powder was obtained by mixing powders containing 80% by weight of zirconia, 20% by weight of alumina, and less than 1% by weight of the other additives, with respect to the whole composite material. Comparative Example 2 having an average particle size of 100 nm was obtained, using the same molding method as in Comparative Example 1.

In Example 1, a mixed powder was obtained by mixing powders containing 50% by weight of zirconia, 50% by weight of alumina, and less than 1% by weight of the other additives, with respect to the whole composite material. Example 1 having a particle size distribution of 60% or more and an average particle size of 100 nm was obtained, using the same molding method as in Comparative Example 1.

In Example 2, a mixed powder was obtained by mixing powders containing 50% by weight of zirconia, 50% by weight of alumina, and less than 1% by weight of other additives, with respect to the whole composite material, and the mixed powder was sintered at 1550° C. for 5 hours under normal pressure using yttrium oxide that is a stabilizer as the other additives to obtain a composite material having a particle size distribution of 60% or more and an average particle size of 150 nm.

In Example 2, a mixed powder was obtained by mixing powder containing 50% by weight of zirconia, powder containing 50% by weight of alumina, and powder containing less than 1% by weight of other additives, with respect to the whole composite material, and the mixed powder was sintered at 1530° C. for 5 hours under normal pressure using yttrium oxide that is a stabilizer as the other additives to obtain a composite material having a particle size distribution of 60% or more and an average particle size of 150 nm.

In Example 3, a mixed powder was obtained by mixing powders containing 65% by weight of zirconia, 35% by weight of alumina, and less than 1% by weight of other additives, with respect to the whole composite material, and the mixed powder was sintered at 1600° C. for 5 hours under normal pressure using yttrium oxide that is a stabilizer as the other additives to obtain a composite material having a particle size distribution of 60% or more and an average particle size of 200 nm.

In Example 4, a mixed powder was obtained by mixing powders containing 65% by weight of zirconia, 35% by weight of alumina, and less than 1% by weight of other additives, with respect to the whole composite material, and the mixed powder was sintered at 1750° C. for 5 hours under normal pressure using yttrium oxide that is a stabilizer as the other additives to obtain a composite material having a particle size distribution of 60% or more and an average particle size of 250 nm.

In Example 5, a mixed powder was obtained by mixing powders containing 70% by weight of zirconia, 30% by weight of alumina, and less than 1% by weight of other additives, with respect to the whole composite material, and the mixed powder was sintered by the same method as in Example 2 using yttrium oxide that is a stabilizer as the other additives to obtain a composite material having a particle size distribution of 60% or more and an average particle size of 150 nm.

In Example 6, a mixed powder was obtained by mixing powders containing 75% by weight of zirconia, 25% by weight of alumina, and less than 1% by weight of other additives, with respect to the whole composite material, and the mixed powder was sintered by the same method as in Example 3 using yttrium oxide that is a stabilizer as the other additives to obtain a composite material having a particle size distribution of 60% or more and an average particle size of 200 nm.

In order to evaluate the wear resistance and strength of the composite material, a pin-on-disc test was performed under conditions of different disc rotation speeds (60 mm/sec, 120 mm/sec) and load applied to the pin (20 N to 120 N) in a state where a pin using distilled water as a lubricant is placed on a circumferential portion of a disc at a distance of 22 mm from the center of the disc. The slide distance was constant (25 km). Considering that the diameter of a tip of the pin is 1.5 mm, an initial frictional pressure applied to the tip of the pin was 20 N to 120 N. Further, the test was carried out three times under each condition, and an average value of the three test results was used as data.

### Reliability Evaluation of Composite Materials

TABLE 1
<Reliability evaluation results of composite materials
according to respective embodiments>

| | | | Average | Specific | | | | |
|---|---|---|---|---|---|---|---|---|
| | Composition | | particle | wear rate | Bending | Fatigue | | Elastic |
| | ZrO₂ | Al₂O₃ | size | (mm³/Nm) | strength | failure | Density | modulus |
| | (vol %) | (vol %) | nm | ×10⁻⁷ | MPa | *crack | g/cm² | MPa |
| Comparative | 40 | 60 | 100 | 35 | 350 | ◯ | 2 | 1450 |
| Example 1 | | | | | | | | |
| Comparative | 80 | 20 | 100 | 40 | 1100 | ◯ | 3 | 550 |
| Example 2 | | | | | | | | |
| Example 1 | 50 | 50 | 100 | 0.095 | 610 | Δ | 4 | 650 |
| Example 2 | 50 | 50 | 150 | 0.088 | 630 | Δ | 4 | 665 |
| Example 3 | 65 | 35 | 200 | 0.045 | 800 | X | 5 | 870 |
| Example 4 | 65 | 35 | 1.250 | 0.085 | 650 | Δ | 4 | 685 |
| Example 5 | 70 | 30 | 150 | 0.030 | 850 | X | 5 | 850 |
| Example 6 | 75 | 25 | 200 | 0.175 | 630 | Δ | 4 | 670 |

*Presence or absence of crack: “◯”-Visible cracks, “Δ”-Microscopic cracks observed under a microscope, and “X”-No crack, In the artificial hip joint including the head 10 and the liner 20, at least one of the head 10 or the liner 20 is made of a composite material including zirconia as a first phase and alumina as a second phase, in which the content of the first phase may be in a range of 50 to 75% by weight, and the content of the second phase may be in a range of 25 to 50% by weight. As can be seen from the results of Table 1, zirconia (ZrO2) has high strength and failure toughness, but its surface hardness is lower than that of alumina (Al2O3).

Thus, by mixing alumina to zirconia to form a composite, it is possible to obtain sufficient surface hardness. In the case of Comparative Example 2 in which the zirconia content is less than 50% by weight, the specific wear rate was high, and the bending strength was 600 MPa or greater, but cracks were seen visually during the fatigue failure test. On the other hand, the elastic modulus, which is the shock absorption capacity, was 1450 MPa, which was very high. In the case of Comparative Example 1 in which the alumina content of is greater than 50% by weight, the specific wear rate was high compared to the examples, the bending strength was 600 MPa or less, and the elastic modulus was 550 MPa or less. Here, in a case where the elastic modulus is less than 600 MPa, deformation of the material becomes large while providing the stress for sufficient shock absorption capacity and there is a risk of adversely affecting the stability of the stem, whereas in a case where the elastic modulus is greater than 1400 MPa, the shock absorption capacity is insufficient and excessive stress is applied to the bone, which results in bone resorption, or the like.

Accordingly, in a case where the content of zirconia is less than 50% by weight or the content of alumina exceeds 50% by weight, there is a concern that sufficient effects may not be obtained in improving the wear resistance of composite ceramics and increasing the strength of zirconia particles.

In particular, in a case where the alumina content exceeds 50% by weight, there is a tendency for alumina particles to sinter with each other, and as a result, there is a concern that the variation in strength of the composite ceramics may increase and the toughness of the composite ceramics may significantly decrease.

Further, in a case where the zirconia content is less than 50% by weight, in the case of phase transition toughness-enhanced ceramics such as zirconia, since a wake zone in which compressive stress is applied to a crack tip is lost under cyclic loading, which results in reduction of the toughness to cause fatigue. In addition, in a case where the zirconia content exceeds 75% by weight, according to results of a dry wear test, and results of a wear track shape on a disc specimen according to the number of cycles and the wear loss of a pin specimen, debris occurs in the whole number of cycles, and there is a concern that the wear loss increases as the number of cycles increases.

Here, each of the composites of Examples 1, 2, 4 and 6 in which the ratio of zirconia to alumina is 50 to 75% by weight to 25 to 50% by weight has a specific wear rate of 1×10⁻⁸ mm³/Nm or less, a bending strength of at least 600 MPa or more, a density of 4 g/cm² or more, and an elastic modulus of 600 MPa. That is, as the weak toughness of alumina is strengthened and the hardness of zirconia is strengthened, it is possible to enhance the strength, failure toughness, workability, and specific wear rate, to thereby extend the implantation life.

As shown in Table 1, particularly, each of Example 3 and Example 5, in which the zirconia content is in a range of 65 to 70% by weight and the alumina content is in a range of 30 to 35% by weight, the particle size distribution of the composite material is 60% or more, and the average particle size is in a range of 150 nm to 200 nm, has a specific wear rate of 0.5×10⁻⁸ mm³/Nm or less, a bending strength of at least 800 MPa, a density of 5 g/cm² or more, a range of an elastic modulus of 800 to 1000 MPa, and no fatigue failure cracks.

Here, in a case where the particle size distribution of the composite is less than 60%, since the particles are uneven, there is a concern that the handling of the powder in the manufacturing process may be difficult. In a case where the average particle size is less than 150 nm, it is difficult to sufficiently densify the composite ceramics, and at the same time, the handling of the raw material powder in the manufacturing process may be significantly difficult. In addition, in a case where the average particle size exceeds 200 nm, there is a concern that zirconia particles or alumina particles are likely to fall off during use of the artificial joint in vivo over a long period of time, and the specific wear rate is rapidly reduced. Accordingly, in a case where the particle size is in the range of 150 nm to 200 nm, it is possible to enhance the specific wear rate to significantly reduce the particle fall-off from the slide surface of the super-mirror-polished artificial joint, so that mechanical properties of the composite ceramics can be stabilized, thereby obtaining high reliability as materials for artificial joints.

### Comparative Examples 3 to 6 and Examples 5 to 8 According to Silicon Nitride and Zirconia

In Comparative Example 3, a mixed powder was obtained by mixing powders containing 95% by weight of silicon nitride, 5% by weight of zirconia, and less than 1% by weight of other additives (including at least one of titania, calcia or cerium oxide), with respect to the whole composite material. The mixed powder was molded by uniaxial pressing and isostatic pressing to obtain a green compact of a predetermined shape, and then, the green compact was sintered at 1400° C. for 5 hours under normal pressure in the air. Accordingly, a specimen having an average particle size of 100 nm was prepared.

In Comparative Example 4, a mixed powder was obtained by mixing powders containing 58 by weight of silicon nitride, 95% by weight of zirconia, and less than 1% by weight of the other additives, with respect to the whole composite material. The mixed power was molded by the same molding method as in Comparative Example 3, and a specimen having an average particle size of 100 nm was prepared.

In Comparative Example 5, a mixed powder was obtained by mixing powders containing 10% by weight of silicon nitride, 90% by weight of zirconia, and less than 1% by weight of other additives (including at least one of titania, calcia and cerium oxide), with respect to the whole composite material. The mixed powder was molded by uniaxial pressing and isostatic pressing to obtain a green compact of a predetermined shape, and then, the green compact was sintered at 1400° C. for 5 hours under normal pressure in the air. Accordingly, a specimen having an average particle size of 100 nm was prepared.

In Comparative Example 6, a mixed powder was obtained by mixing powders containing 90% by weight of silicon nitride, 10% by weight of zirconia, and less than 1% by weight of the other additives, with respect to the whole composite material. The mixed power was molded by the same molding method as in Comparative Example 3, and a specimen having an average particle size of 100 nm was prepared.

In Example 5, a mixed powder was obtained by mixing powders containing 10% by weight of silicon nitride, 90% by weight of zirconia, and less than 1% by weight of other additives, with respect to the whole composite material, and the mixed powder was sintered at 1400° C. for 5 hours under normal pressure using yttrium oxide that is a stabilizer as the other additives to obtain a composite material having a particle size distribution of 60% or more and an average particle size of 100 nm. A specimen was prepared in which porous grooves having a diameter or lateral size of 100 μm to 400 μm in the maximum micropore, a ratio of the depth to the lateral size of 0.04 to 0.2, and an area density of the micropores of 1% to 30% were formed on the inner surface of the liner 20 and the outer surface of the head 10 facing the liner 20, formed of the composite material, respectively.

In Example 6, a mixed powder was obtained by mixing powders containing 50% by weight of silicon nitride, 50% by weight of zirconia, and less than 1% by weight of other additives, with respect to the whole composite material, and the mixed powder was sintered at 1530° C. for 5 hours under normal pressure using yttrium oxide that is a stabilizer as the other additives to obtain a composite material having a particle size distribution of 60% or more and an average particle size of 150 nm. A specimen was prepared in which porous grooves having a diameter or lateral size of 50 μm to 100 μm in the maximum micropore, a ratio of the depth to the lateral size of 0.05 to 0.18, and an area density of the micropores of 1% to 30% were formed on the inner surface of the liner 20 and the outer surface of the head 10 facing the liner 20, formed of the composite material, respectively.

In Example 7, a mixed powder was obtained by mixing powders containing 90% by weight of silicon nitride, 10% by weight of zirconia, and less than 1% by weight of other additives, with respect to the whole composite material, and the mixed powder was sintered at 1600° C. for 5 hours under normal pressure using yttrium oxide that is a stabilizer as the other additives to obtain a composite material having a particle size distribution of 60% or more and an average particle size of 200 nm, and porous grooves of the same micropore range as in Example 2 were formed in each of the head 10 and the liner 20 made of the composite material.

In Example 8, a mixed powder was obtained by mixing powder containing 90% by weight of silicon nitride, powder containing 10% by weight of zirconia, and less than 1% by weight of other additives, with respect to the whole composite material, and the mixed powder was sintered at 1750° C. for 5 hours under normal pressure using yttrium oxide that is a stabilizer as the other additives to obtain a composite material having a particle size distribution of 60% or more and an average particle size of 250 nm, and porous grooves of the same micropore range as in Example 1 were formed in each of the head 10 and the liner 20 made of the composite material.

In order to evaluate the wear resistance and strength of the composite material, a pin-on-disc test was performed under conditions of different disc rotation speeds (60 mm/sec, 120 mm/sec) and load applied to the pin (20 N to 120 N) in a state where a pin using distilled water as a lubricant is placed on a circumferential portion of a disc at a distance of 22 mm from the center of the disc. The slide distance was constant (25 km). Considering that the diameter of a tip of the pin is 1.5 mm, an initial frictional pressure applied to the tip of the pin was 20 N to 120 N. Further, the test was carried out three times under each condition, and an average value of the three test results was used as data.

### Reliability Evaluation Test

TABLE 2
<Reliability evaluation results of composites according to respective examples>

| | | | Surface | Average | Specific | | | | |
|---|---|---|---|---|---|---|---|---|---|
| | Composition | | porosity | particle | wear rate | Bending | Fatigue | | Elastic |
| | Si₃N₄ | ZrO₂ | Presence/ | size | (mm³/Nm) | strength | failure | Density | modulus |
| | (vol % ) | (vol %) | absence | nm | ×10⁻⁷ | MPa | *crack | g/cm² | MPa |
| Comparative | 95 | 5 | X | 100 | 35 | 1100 | ◯ | 3 | 350 |
| Example 3 | | | | | | | | | |
| Comparative | 5 | 95 | X | 100 | 40 | 350 | ◯ | 3 | 450 |
| Example 4 | | | | | | | | | |
| Comparative | 10 | 90 | X | 100 | 1.5 | 650 | ◯ | 3 | 570 |
| Example 5 | | | | | | | | | |
| Comparative | 90 | 10 | X | 100 | 2.3 | 660 | ◯ | 3 | 585 |
| Example 6 | | | | | | | | | |
| Example 5 | 10 | 90 | ◯ | 100 | 0.095 | 610 | Δ | 4 | 650 |
| Example 6 | 50 | 50 | ◯ | 150 | 0.038 | 830 | X | 5 | 820 |
| Example 7 | 90 | 10 | ◯ | 200 | 0.045 | 800 | X | 5 | 870 |
| Example 8 | 90 | 10 | ◯ | 250 | 0.085 | 650 | Δ | 4 | 685 |

*Presence or absence of crack: “◯”-Visible cracks, “Δ”-Microscopic cracks observed under a microscope, and “X”-No crack

As can be seen from the results of Table 2, in the case of Comparative Example 4 having a silicon nitride content of less than 10% by weight, the specific wear rate was higher and the elastic modulus was lower than those of the other examples. Further, anti-pathogenic properties of silicon nitride are inferior, and there is a limitation that X-ray, CT and MRI cannot be easily seen without image distortion. In addition, the advantage of silicon nitride of high hardness disappears.

On the other hand, in the case of Comparative Example 3 having a zirconia content of less than 10% by weight, the specific wear rate was high, the bending strength was low, and the elastic modulus was low compared to the other examples. Here, in a case where the elastic modulus is less than 600 MPa, deformation of the material becomes large while providing the stress for sufficient shock absorption capacity and there is a risk of adversely affecting the stability of the stem, whereas in a case where the elastic modulus is greater than 1400 MPa, the shock absorption capacity is insufficient and excessive stress is applied to the bone, which results in bone resorption, or the like.

Further, in each of Comparative Example 3 and Comparative Example 4, the occurrence of cracks during the fatigue failure test was visually observed. In other words, in the case of phase transition toughness-enhanced ceramics such as zirconia, since a wake zone in which compressive stress is applied to a crack tip is lost under cyclic loading, which results in reduction of the toughness to cause fatigue.

In the artificial hip joint including the head 10 and the liner 20, at least one of the head 10 or the liner 20 is made of a composite material including silicon nitride (Si3N4) as a first phase and zirconia (ZrO2) as a second phase, in which the content of the first phase may be in a range of 10 to 95% by weight, and the content of the second phase may be in a range of 90 to 10% by weight. Accordingly, each of the composite ceramics of Examples 5 to 8, having a ratio of silicon nitride to zirconia (10˜90% by weight to 10˜90% by weight), has a specific wear rate of 1×10-8 mm³/Nm or less, a bending strength of at least 600 MPa or more, a density of 4 g/cm² or more, and an elastic modulus of 600 MPa.

In the head 10 and the liner 20, the particle size distribution of the composite may be 60% or more, and/or the average particle size may be in the range of 150 nm to 200 nm.

In a case where the particle size distribution of the composite is less than 60%, since the particles are uneven, there is a concern that the handling of the powder in the manufacturing process may be difficult. In a case where the average particle size is less than 150 nm, it is difficult to sufficiently densify the composite ceramics, and at the same time, the handling of the raw material powder in the manufacturing process may be significantly difficult. In addition, in a case where the average particle size exceeds 200 nm, there is a concern that zirconia particles or silicon nitride particles in the particle system are likely to fall off, thereby making it difficult to obtain a super-mirror surface. Further, there is a concern that zirconia or silicon nitride particles are likely to fall off during use of the artificial joint in vivo over a long period of time, and the specific wear rate is rapidly reduced. Accordingly, in a case where the average particle size is in the range of 150 nm to 200 nm, it is possible to enhance the wear resistance, and to significantly reduce the particle fall-off from the slide surface of the super-mirror-polished artificial joint, so that mechanical properties of the composite ceramics can be stabilized, thereby obtaining high reliability as materials for artificial joints.

As shown in the results of Table 2, particularly, each of Example 6 and Example 7 has a specific wear rate of 0.5×10-8 mm3/Nm or less, a bending strength of at least 800 MPa, a density of 5 g/cm2 or more, a range of an elastic modulus of 800 to 1000 MPa, and no fatigue failure cracks.

Further, the head 10 and the liner 20 may have a surface roughness value of 0.01 μm±50% and a roundness value of 0.1 μm±50% in a pin-on-disc wear test. In a case where the head 10 and the liner 20 have the above-mentioned surface roughness and roundness values, the friction coefficient becomes low to achieve smooth sliding coupling, thereby making it possible to obtain a very low specific wear rate.

### Comparative Examples 1, 4 and Examples 9 to 12 According to Composite and Single Materials

In Comparative Example 1, a mixed powder was obtained by mixing powders containing 60% by weight of alumina, 40% by weight of zirconia, and less than 1% by weight of other additives (including at least one of titania, calcia or cerium oxide), with respect to the whole composite material. The mixed powder was molded by uniaxial pressing and isostatic pressing to obtain a green compact of a predetermined shape, and then, the green compact was sintered at 1400° C. for 5 hours under normal pressure in the air. Accordingly, a specimen having an average particle size of 100 nm was prepared.

In Comparative Example 4, a mixed powder was obtained by mixing powders containing 5% by weight of silicon nitride, 95% by weight of zirconia, and less than 18 by weight of the other additives, with respect to the whole composite material. The mixed power was molded by the same molding method as in Comparative Example 1, and a specimen having an average particle size of 100 nm was prepared.

In Example 9, a mixed powder was obtained by mixing powders containing 100% by weight of silicon nitride and less than 1% by weight of other additives, and the mixed powder was sintered at 1400° C. for 5 hours under normal pressure using yttrium oxide that is a stabilizer as the other additives to obtain a specimen having a particle size distribution of 60% or more and an average particle size of 100 nm. The inner surface of the liner 20 and the outer surface of the head 10 facing the liner 20, molded by the specimen, respectively, were formed with dimple grooves having a diameter or lateral size of 100 μm to 400 μm in the maximum micropore, a ratio of the depth to the lateral size of 0.04 to 0.2, and an area density of the micropores of 1% to 30%.

In Example 10, a mixed powder was obtained by mixing powders containing 100% by weight of silicon nitride and less than 1% by weight of other additives, and the mixed powder was sintered at 1530° C. for 5 hours under normal pressure using yttrium oxide that is a stabilizer as the other additives to obtain a specimen having a particle size distribution of 60% or more and an average particle size of 150 nm. The inner surface of the liner 20 and the outer surface of the head 10 facing the liner 20, molded by the specimen, respectively, were formed with dimple grooves having a diameter or lateral size of 50 μm to 100 μm in the maximum micropore, a ratio of the depth to the lateral size of 0.05 to 0.18, and an area density of the micropores of 1% to 30%.

In Example 11, a mixed powder was obtained by mixing powders containing 100% by weight of silicon nitride and less than 1% by weight of other additives, and the mixed powder was sintered at 1600° C. for 5 hours under normal pressure using yttrium oxide that is a stabilizer as the other additives to obtain a specimen having a particle size distribution of 60% or more and an average particle size of 200 nm, and then, dimple grooves having the same micropore range as in Example 10 were formed on the head 10 and the liner 20 made of the specimen.

In Example 12, a mixed powder was obtained by mixing powders containing 100% by weight of silicon nitride and less than 1% by weight of other additives, and the mixed powder was sintered at 1750° C. for 5 hours under normal pressure using yttrium oxide that is a stabilizer as the other additives to obtain a specimen having a particle size distribution of 60% or more and an average particle size of 250 nm, and then, dimple grooves having the same micropore range as in Example 9 were formed on the head 10 and the liner 20 made of the specimen.

In order to evaluate the wear resistance and strength of the specimen, a pin-on-disc test was performed under conditions of different disc rotation speeds (60 mm/sec, 120 mm/sec) and load applied to the pin (20 N to 120 N) in a state where a pin using distilled water as a lubricant is placed on a circumferential portion of a disc at a distance of 22 mm from the center of the disc. The slide distance was constant (25 km). Considering that the diameter of a tip of the pin is 1.5 mm, an initial frictional pressure applied to the tip of the pin was 20 N to 120 N. Further, the test was carried out three times under each condition, and an average value of the three test results was used as data.

### Reliability Evaluation Test

TABLE 3
<Reliability evaluation results according to respective embodiments>

| | | | | Surface | Maximum | Average | Specific | | | | |
|---|---|---|---|---|---|---|---|---|---|---|---|
| | Composition | | | dimple | micropore | particle | wear rate | Bending | Fatigue | | Elastic |
| | Al₂O₃ | Si₃N₄ | ZrO₂ | Presence/ | diameter | size | (mm³/Nm) | strength | failure | density | modulus |
| | vol % | vol % | vol % | absence | μm | nm | ×10⁻⁷ | MPa | *crack | g/cm² | MPa |
| Comparative | 60 | — | 40 | X | — | 100 | 35 | 1100 | ◯ | 3 | 550 |
| Example 1 | | | | | | | | | | | |
| Comparative | — | 5 | 95 | X | — | 100 | 40 | 350 | ◯ | 3 | 350 |
| Example 4 | | | | | | | | | | | |
| Example 9 | — | 100 | — | ◯ | 100~400 | 100 | 0.095 | 610 | Δ | 4 | 650 |
| Example 10 | — | 100 | — | ◯ | 50~100 | 150 | 0.038 | 830 | X | 5 | 820 |
| Example 11 | — | 100 | — | ◯ | 50~100 | 200 | 0.045 | 800 | X | 5 | 870 |
| Example 12 | — | 100 | — | ◯ | 100~400 | 250 | 0.085 | 650 | Δ | 4 | 685 |

*Presence or absence of crack: “◯”-Visible cracks, “Δ”-Microscopic cracks observed under a microscope, and “X”-No crack

As can be seen from the results of Table 3, in each case of Comparative Example 1 and Comparative Example 4, the specific wear rate was high, the bending strength was too high or too low, and the elastic modulus was lower than those of the other examples. Here, in a case where the elastic modulus is less than 600 MPa, deformation of the material becomes large while providing the stress for sufficient shock absorption capacity and there is a risk of adversely affecting the stability of the stem, whereas in a case where the elastic modulus is greater than 1400 MPa, the shock absorption capacity is insufficient and excessive stress is applied to the bone, which results in bone resorption, or the like.

Further, in each of Comparative Example 1 and Comparative Example 4, the occurrence of cracks during the fatigue failure test was visually observed. In other words, in the case of phase transition toughness-enhanced ceramics such as zirconia, since a wake zone in which compressive stress is applied to a crack tip is lost under cyclic loading, which results in reduction of the toughness to cause fatigue.

Accordingly, each of Examples 9 to 12 has a specific wear rate of 1×10-8 mm3/Nm or less, a bending strength of at least 600 MPa or more, a density of 4 g/cm2 or more, and an elastic modulus of 600 to 1000 MPa.

In the head 10 and the liner 20, the particle size distribution of the silicon nitride may be 60% or more, and the average particle size of the silicon nitride may be in the range of 150 nm to 200 nm. In a case where the particle size distribution of the silicon nitride is less than 60%, since the particles are uneven, there is a concern that the handling of the powder in the manufacturing process may be difficult. In a case where the average particle size is less than 150 nm, it is difficult to sufficiently densify the ceramics, and at the same time, the handling of the raw material powder in the manufacturing process may be significantly difficult. Further, in a case where the average particle size exceeds 200 nm, there is a concern that silicon nitride particles in the particle system are likely to fall off, thereby making it difficult to obtain a super-mirror surface. In addition, there is a concern that particle fall-off is likely to occur during use of the artificial joint in vivo over a long period of time, and the wear resistance is rapidly reduced. Accordingly, in a case where the average particle size is in the range of 150 nm to 200 nm, it is possible to enhance the wear resistance to significantly reduce the particle fall-off from the slide surface of the super-mirror-polished artificial joint, so that mechanical properties of the ceramics can be stabilized, thereby obtaining high reliability as materials for artificial joints.

As shown in the results of Table 1, each of Example 10 and Example 11, compared to each of Example 9 and Example 12, has a specific wear rate of 0.5×10-8 mm3/Nm or less, a bending strength of 800 MPa or more, a density of 5 g/cm2 or more, and a range of an elastic modulus of 800 to 1000 MPa, and shows no visible fatigue failure cracks.

Further, the head 10 and the liner 20 may have a surface roughness value of 0.01 μm±50% and a roundness value of 0.1 μm±50% in a pin-on-disc wear test. In a case where the head 10 and the liner 20 have the above-mentioned surface roughness and roundness values, the friction coefficient becomes low to achieve smooth sliding coupling, thereby making it possible to obtain a very low specific wear rate.

### Biocompatibility Evaluation Test

As a result of rat macrophage experiments in which the artificial hip joint was contacted with a rat macrophage RAW 264.7 cell line for 24 hours, a cell survival rate of 70% or more was obtained, and in relation to inflammatory response, the amount of production of TNF-α was 300 picograms/ml or less, and the amount of production of IL-1 was 50 picograms/ml or less. Accordingly, it was confirmed that the artificial hip joint according to the present invention has high biocompatibility through the above-mentioned characteristics, thereby making it possible to prevent inflammatory reactions. Further, it was found that macrophage response, macrophage stability, and cytokine production were superior to those of other ceramic materials.

### SARS-CoV-2 Virus Inactivation Test

As a result of investigation of fragmentation of virus RNA upon 1-minute contact with the powder through RT-PCR experiments on virions N-gene sequences, fluorescent micrographs were obtained in which viral proteins, F-actin, and cell nuclei were respectively visualized as red, green, and blue stains by performing staining and then performing inoculation of VeroE6/TMPRSS2 cells. Through quantification of fluorescence microscopy data provided as the percentage of infected cells to total cells, that is, the percentage of red stained cells to the total blue stained nuclei and the percentage of viable cells to the total cells, it was found that the SARS-CoV-2 virus was inactivated on the head surface formed of silicon nitride.

### Surface Bacterial Adhesion Inhibition Test

As a result of performing MBC comparison of various antimicrobials against airborne bacteria and biofilm-forming bacteria in a surface medium of the head formed of silicon nitride, the resistance of the biofilm-forming bacteria according to the biofilm formation time did not change. Accordingly, the head formed of silicon nitride is advantageous to human cells, and has effects of promoting osseointegration and inhibiting bacterial adhesion to the surface.

Further, the head 10 may be made of 100% by weight of silicon nitride (Si3N4), and the outer surface of the head 10 may be formed with dimples. Silicon nitride has the advantages of implant stability, anti-pathogenic properties, and an image without distortion in X-ray, CT and MRI. In addition, it is possible to enhance lubricity to the liner 20, thereby reducing a frictional force to enhance wear resistance performance.

The above-mentioned embodiments are only exemplary, and various modifications and equivalent other embodiments may be made therefrom by those skilled in the art. Accordingly, the true technical protection scope of the present invention should be determined by the technical spirit of the invention described in the claims.

1. An artificial hip joint comprising a head and a linear,
wherein at least one of the head or the liner is formed of a composite containing zirconia as a first phase and alumina as a second phase,
wherein the content of the first phase is in a range of 65 to 70% by weight, and
wherein the content of the second phase is in a range of 30 to 35% by weight,
wherein the particle size distribution of the composite is 60% or more, and the average particle size of the composite is in a range of 150 nm to 200 nm,
wherein each of the head and the liner satisfies all of Conditional Expression 1,
$${R}_{H} > {L}_{a 1} \qquad [ Conditional expression 1 ]$$
$${R}_{H} = {L}_{a 2}$$
$${R}_{a} \leq 0.05 μm$$
where R_{H} is the outer diameter of the head, Lₐ₁ is the groove diameter of the top inner surface of the liner, Lₐ₂ is the groove diameter of the inner surface of the liner, and Rₐ is an arithmetic mean surface roughness, and
wherein the head and the liner have a specific wear rate of 0.5×10⁻⁸ mm³/Nm or less, a bending strength of at least 800 MPa or more, a density of 5 g/cm² or more, and an elastic modulus in a range of 800 to 1000 MPa, and show no fatigue failure cracks.

2. (canceled)

3. An artificial hip joint comprising a head and a linear,
wherein at least one of the head or the liner is formed of a composite containing silicon nitride as a first phase and zirconia as a second phase,
wherein the content of the first phase is in a range of 10 to 90% by weight,
wherein the content of the second phase is in a range of 90 to 10% by weight,
wherein the particle size distribution of the composite is 60% or more, and the average particle size of the composite is in a range of 150 nm to 200 nm,
wherein each of the head and the liner satisfies all of Conditional Expression 2,
$${R}_{H} > {L}_{a 1} \qquad [ Conditional expression 2 ]$$
$${R}_{H} = {L}_{a 2}$$
$${R}_{a} \leq 0.05 μm$$
where R_{H} is the outer diameter of the head, Lₐ₁ is the groove diameter of the top inner surface of the liner, Lₐ₂ is the groove diameter of the inner surface of the liner, and Rₐ is an arithmetic mean surface roughness, and
wherein the head and the liner have a specific wear rate of 0.5×10⁻⁸ mm³/Nm or less, a bending strength of at least 800 MPa or more, a density of 5 g/cm² or more, and an elastic modulus in a range of 800 to 1000 MPa, and show no fatigue failure cracks.

4. (canceled)

5. (canceled)

6. An artificial hip joint comprising a head and a liner,
wherein the head contains 100% by weight of silicon nitride,
wherein the particle size distribution of the silicon nitride is 60% or more, and the average particle size of the silicon nitride is in a range of 150 nm to 200 nm,
wherein each of the head and the liner satisfies all of Conditional Expression 3,
$${R}_{H} > {L}_{a 1} \qquad [ Conditional expression 3 ]$$
$${R}_{H} = {L}_{a 2}$$
$${R}_{a} \leq 0.05 μm$$
where R_{H} is the outer diameter of the head, Lₐ₁ is the groove diameter of the top inner surface of the liner, Lₐ₂ is the groove diameter of the inner surface of the liner, and Rₐ is an arithmetic mean surface roughness, and
wherein the head and the liner have a specific wear rate of 0.5×10⁻⁸ mm³/Nm or less, a bending strength of at least 800 MPa or more, a density of 5 g/cm² or more, and an elastic modulus in a range of 800 to 1000 MPa, and show no fatigue failure cracks.

7. (canceled)

8. The artificial hip joint according to claim 6,
wherein any one of the head and liner is configured so that SARS-CoV-2 virus is inactivated.

9. The artificial hip joint according to claim 1,
wherein at least one of an inner surface of the liner or an outer surface of the head facing the liner is formed with a plurality of dimples having a predetermined size and a predetermined depth.

10. The artificial hip joint according to claim 9,
wherein the diameter or lateral size of the maximum micropore among the dimples is in a range of 100 μm to 400 μm, the ratio of the depth to the lateral size is in a range of 0.04 to 0.2, and the area density of the micropores is 1% to 30%.

11. The artificial hip joint according to claim 9,
wherein the diameter or lateral size of the minimum micropore of the dimples is in a range of 50 μm to 100 μm, the ratio of the depth to the lateral size is in a range of 0.05 to 0.18, and the area density of the micropores is 1% to 30%.

12. (canceled)

13. (canceled)

14. The artificial hip joint according to claim 1,
wherein the head has a surface roughness value of 0.01 μm±50% and a roundness value of 0.1 μm±50%.

15. The artificial hip joint according to claim 1,
wherein any one of the head and liner is configured so that as a result a rat macrophage experiment in which the artificial hip joint is contacted with a rat macrophage RAW 264.7 cell line for 24 hours, a cell survival rate of 70% or more is obtained, and in relation to inflammatory response, the amount of production of TNF-α is 300 picograms/ml or less, and the amount of production of IL-1 is 50 picograms/ml or less.

16. The artificial hip joint according to claim 1,
wherein the liner is formed with a groove portion at an upper apex thereof.

17. The artificial hip joint according to claim 3,
wherein at least one of an inner surface of the liner or an outer surface of the head facing the liner is formed with a plurality of dimples having a predetermined size and a predetermined depth.

18. The artificial hip joint according to claim 17,
wherein the diameter or lateral size of the maximum micropore among the dimples is in a range of 100 μm to 400 μm, the ratio of the depth to the lateral size is in a range of 0.04 to 0.2, and the area density of the micropores is 1% to 30%.

19. The artificial hip joint according to claim 17,
wherein the diameter or lateral size of the minimum micropore of the dimples is in a range of 50 μm to 100 μm, the ratio of the depth to the lateral size is in a range of 0.05 to 0.18, and the area density of the micropores is 1% to 30%.

20. The artificial hip joint according to claim 3,
wherein the head has a surface roughness value of 0.01 μm±50% and a roundness value of 0.1 μm±50%.

21. The artificial hip joint according to claim 3,
wherein any one of the head and liner is configured so that as a result a rat macrophage experiment in which the artificial hip joint is contacted with a rat macrophage RAW 264.7 cell line for 24 hours, a cell survival rate of 70% or more is obtained, and in relation to inflammatory response, the amount of production of TNF-α is 300 picograms/ml or less, and the amount of production of IL-1 is 50 picograms/ml or less.

22. The artificial hip joint according to claim 3,
wherein the liner is formed with a groove portion at an upper apex thereof.

23. The artificial hip joint according to claim 6,
wherein at least one of an inner surface of the liner or an outer surface of the head facing the liner is formed with a plurality of dimples having a predetermined size and a predetermined depth.

24. The artificial hip joint according to claim 23,
wherein the diameter or lateral size of the maximum micropore among the dimples is in a range of 100 μm to 400 μm, the ratio of the depth to the lateral size is in a range of 0.04 to 0.2, and the area density of the micropores is 1% to 30%.

25. The artificial hip joint according to claim 23,
wherein the diameter or lateral size of the minimum micropore of the dimples is in a range of 50 μm to 100 μm, the ratio of the depth to the lateral size is in a range of 0.05 to 0.18, and the area density of the micropores is 1% to 30%.

26. The artificial hip joint according to claim 6,
wherein the head has a surface roughness value of 0.01 μm±50% and a roundness value of 0.1 μm±50%.

27. The artificial hip joint according to claim 6,
wherein any one of the head and liner is configured so that as a result a rat macrophage experiment in which the artificial hip joint is contacted with a rat macrophage RAW 264.7 cell line for 24 hours, a cell survival rate of 70% or more is obtained, and in relation to inflammatory response, the amount of production of TNF-α is 300 picograms/ml or less, and the amount of production of IL-1 is 50 picograms/ml or less.

28. The artificial hip joint according to claim 6,
wherein the liner is formed with a groove portion at an upper apex thereof.
